(19) United States
(12) Patent Application Publication
(10) Pub. No.: US 2010/0152645 A1
(43) Pub. Date: Jun. 17, 2010

(54) Facial Hair-Growth Device and Facial Hair-Growth System

(76) Inventor: Masahiro Ogasawara, Osaka (JP)

Correspondence Address: OGILVIE LAW FIRM, 2552 South Wilshire Circle, SALT LAKE CITY, UT, 84109, US

(21) Appl. No.: 12/522,531

(22) PCT Filed: Jan. 4, 2008
(86) PCT No.: PCT/JP2008/050002

§ 371 Date: Feb. 23, 2010

(30) Foreign Application Priority Data
Jan. 9, 2007 (JP) 2007-001538
Jan. 9, 2007 (JP) 2007-001539

(51) Int. Cl. A61N 5/06; A61N 1/30

(52) U.S. Cl. 604/20; 607/88

(57) ABSTRACT

Compared to a conventional halogen lamp and optical filter, wavelength range of a light is narrowed, throughput of a desired wavelength range is increased, and irradiation of a light of a different wavelength range is enabled. The present apparatus for facial treatment and hair restoration is a combination of a light source means, which is a light source of a monochromatic light of an ultra narrow bandwidth having a half bandwidth of 10 nm or less emitted from a monochromatic LED light source, a monochromatic laser diode light source, or a laser light source, and a diffusion means. Specifically, in a case where the LED light source is used, the apparatus includes a bandpass filter for focusing the wavelength range of each light emitted from the light source and a diffusion lens for diffusing a light which passed through the filter and has an ultra narrow bandwidth with a half bandwidth of 10 nm or less.

### TECHNICAL FIELD

The present invention is facial treatment/hair restoration device that irradiates light to the skin, and it relates to the facial treatment/hair restoration device that can irradiate the light of desired wavelength. In particular, the monochromatic light of a super-narrowband can be irradiated. Ultimately, it relates to the facial treatment/hair restoration device and system that can adjust the peak wave length without stage.

### BACKGROUND ART

Conventionally, in the optical facial treatment device which irradiates the facial skin with the light of the visible light region except an infrared and ultraviolet wavelength band, it irradiates with the light of the visible light region by attaching the filter that penetrates only specific wave length light of halogen lamp. Moreover, in other optical facial treatment device, what combines monochrome LED (Light Emitting Diode) and irradiates the skin with the excitation light of a desired wavelength band region is known (patent documents 1).

Moreover, the laser (the product name “Revage670”) for the hair restoration only authorized by United States FDA is known (See non-patent documents 1). This arranged plural red laser diodes of the wavelength of 670 nm. By irradiating light to the skin while scanning, the follicle of pile of the resting phase is induced at the term of growth. However, in the one to irradiate to the skin by arranging plural red laser diodes and by scanning, there is a serious problem that it cannot irradiate it to the entire diseased part at a time.

While confirming the efficacy of the above-mentioned optical facial treatment device, this inventor thought that width of wavelength of the irradiating light narrow, excellent in monochrome one has an excellent effect in facial treatment/hair restoration. And, plural devices that were able to actually irradiate monochromatic light that the width of wavelength of the irradiating light is narrowly excellent in monochrome were prepared. And, the effect was confirmed. A final target is to produce the facial treatment/hair restoration device that can adjust, to a no stage, the peak wave length of monochromatic light that the width of wavelength is narrowly excellent in monochrome as the monochromatic light of the best wavelength band can be irradiated considering the individual variation in the effect of the facial treatment/hair restoration.

First of all, the means to take out monochromatic light with narrow width of wavelength, cutting wavelength of light of lamp source of light with filter, was discussed. This is the means of transmitting only necessary wavelength from white light such as xenon lamps and halogen lamps including wide-ranging wavelength element from an optical outside purple region to an infrared rays optical region, and irradiating. However, there is a problem of describing this means as follows. That is, the following problem is enumerated. Monochrome depends on the filter characteristic used. Permeability decreases by the selection of the filter with a narrow penetration bandwidth to improve monochrome. The filter is necessary only for a necessary wavelength element.

Moreover, the spectrum means of the lamp light with a spectroscope was discussed. This method uses the grating built into spectroscopic. And, white light that contains wide wavelength element from ultraviolet rays to infrared rays optical region like xenon lamp and halogen lamp, etc. is spectrum. Light is divided like the prism by changing the angle of the grating. Monochromatic light can be switched by adjusting a spectroscopic slit width. Moreover, the adjustment such as obtaining quantities of light by lowering a monochrome character is also possible. However, since the spectrum is done by using the grating, monochromatic light that goes out by passing spectroscopic slit weakens very much.

Moreover, vitamins A, C, E and the like are known as indispensable minerals for skin and a technique to cause such vitamins to be efficiently permeated deep into skin to maintain healthy skin has been studied. For example, a technique to give skin ultrasonic wave energy as a facial treatment apparatus or a skin beautifying apparatus or a technique to efficiently cause medicinal properties to be absorbed into skin by use of iontophoresis has been used. A technique for skin beautifying or facial treatment by use of iontophoresis ionizes water-soluble vitamins or the like which are hardly absorbed by skin by ordinary external application so that the vitamins or the like can be absorbed into skin by use of electric current. Moreover, the technique for skin beautifying or facial treatment by use of ultrasonic wave enables introduction of oil-soluble or water-soluble high-molecule properties.

Meanwhile, a method to stimulate hair restoration or hair growth by efficiently activating skin and cells under the skin of the cephalic part or the like of a patient who has less hair in the cephalic part due to aging, stress, heredity, disease, or the like has been demanded (e.g., refer to the Patent Documents 2 and 3). For this purpose, various kinds of liquid-type medical agents are being sold. A method to stimulate the skin by patting on the cephalic part using a brush or the like is also known. However, according to the conventional technique, a method to efficiently enhance hair restoration or hair growth has not been found yet.

Various discussions about mentioning above were done. The device of the composition described as follows was produced, and the effect was confirmed.

[Patent document 1]JP, 2005-000276, A

[Patent document 2]JP, 2001-259045, A

[Patent document 3]JP, 2005-245521, A

[Non-patent documents 1]http://apirascience.com/ REVAGE670 PROVEN LASER HAIR REJUVENATION

### DISCLOSURE OF THE INVENTION

### Problems to be Solved by the Invention

The present invention aims to provide an apparatus for facial treatment and hair restoration which irradiates a monochromatic light of an ultra narrow bandwidth having a half bandwidth of 10 nm or less to an area of wide range and can ultimately carry out almost nonstep control of the peak wavelength of light.

Moreover, the present invention aims to provide a system for facial treatment and hair restoration for enhancing hair restoration and hair growth by activating skin and cells under the skin of the cephalic part or the like for a patient who has less hair in the cephalic part due to aging, stress, heredity, disease, or the like and for carrying out beautification of skin and facial treatment.

### Means to Solve the Objects

The inventor of the present invention is a medical doctor who specializes in cosmetic dermatological treatment, providing information regarding hair restoration, hair growth and prevention of hair loss, and consulting services regarding the above. Through operation of the above, the inventor has keenly studied a method for beautiful skin and for hair restoration and growth which is safe and free from a trouble to skin even if the method is used for a long time and in addition enables to obtain a sufficient effect in a short period of time. The inventor made various prototypes and actually confirmed the effect of the apparatus in his beauty clinic to complete the present invention.

An apparatus for facial treatment and hair restoration of a first aspect of the present invention for achieving the above-mentioned purpose includes a light source means which can emit a monochromatic light of an ultra narrow bandwidth having a half bandwidth of 10 nm or less and a diffusion means which can diffuse the monochromatic light of an ultra narrow bandwidth emitted from the light source means so that an area for irradiation can be increased.

Irradiating the monochromatic light of an ultra narrow bandwidth having a half bandwidth of 10 nm or less on an affected area enables to obtain a stronger effect than irradiating the same volume of light in an approximate wavelength range. That is, it becomes possible to make use of the characteristics of the selected light of an ultra narrow bandwidth for beauty. Irradiating the monochromatic light of an ultra narrow bandwidth having a half bandwidth of 10 nm or less enables to significantly reduce interference by the light from the light source and to give skin and cells under the skin the interactive effects the monochromatic light originally possesses.

Moreover, in case of the above-mentioned red-color laser which has been approved by the US Food and Drug Administration (FDA), the light is irradiated onto an affected area in a spotted manner and is caused to scan the irradiation area two-dimensionally. However, the present invention uses the diffusion means to simultaneously irradiate the light to the affected area. Thus, it becomes possible to carry out irradiation in an area of wide range and to carry out treatment within the shortest period of time. In other words, in the case of the present invention, total irradiation time to skin cells in one spot becomes longer and the light is effectively irradiated so that a remarkably high efficiency ratio can be obtained. Moreover, instead of irradiating light such as a laser beam having a strong energy, a monochromatic light having a weak energy is irradiated on the skin, thereby the facial treatment effect or the hair restoration effect caused by the wavelength is significantly realized.

Next, an apparatus for facial treatment and hair restoration of a second aspect of the present invention is characterized in having comprised a peak wavelength variation means for enabling to vary a peak wavelength of the monochromatic light.

According to such a configuration, it becomes possible to obtain a unique treatment effect for a body response to the wavelength.

A peak wavelength variable means includes, for example, an optical parametric oscillator. A specific example includes a case where a light irradiated from a flash lamp-excited Nd:YAG laser light source is made incident upon a crystal of a light parametric oscillator (e.g., LT-2214 which is broadband OPO of the LOTIS TII) so that the wavelength can be continuously varied. Moreover, another example is a case where the wavelength is continuously varied by use of the Rainbow Laser of the Seiko Electric Co., Ltd. Other than the above, the LUMINATOR® by DRIEL may be used as a means for varying the wavelength. The peak wavelength variable means is used to make the peak wavelength of a monochromatic light continuously variable, thereby making it possible to vary the wavelength enables to establish the best treatment for each person depending on the person and symptoms and ultimately to carry out shiftable nonstep control of the peak wavelength.

Here, it is preferable that the peak wavelength variable means of the apparatus for facial treatment and hair restoration of the second aspect can vary the peak wavelength of the monochromatic light in a red or orange wavelength range. Irradiating a light in a red or orange wavelength range has an effect of warming the surface of the skin. Moreover, irradiating a light in a red or orange wavelength range can increase fibroblast and collagen.

Moreover, it is preferable that the peak wavelength variable means of the apparatus for facial treatment and hair restoration of the second aspect can vary the peak wavelength of the monochromatic light in a green wavelength range.

Moreover, it is preferable that the peak wavelength variable means of the apparatus for facial treatment and hair restoration of the second aspect can vary the peak wavelength of a monochromatic light in the blue wavelength range. By use of the blue wavelength range, the peak wavelength variable means can effectively act on treatment of activated acne with bactericidal action. Roles of each wavelength will be gradually revealed by future research.

Moreover, the apparatus for facial treatment and hair restoration of a first aspect of the present invention include the light source means includes a plurality of monochromatic light emitting diode light sources having different peak wavelengths and a bandpass filter for focusing wavelength bands of each light emitted from the monochromatic light emitting diode light sources so that the light has a half bandwidth of 10 nm or less.

Here, a monochromatic light emitting diode (LED) light source can emit a visible light, which is one or more selected from blue, blue green, green, yellow, orange, and red. The monochromatic LED light source may be a three-color LED of red, blue, and green or a combination of LEDs of other colors. However, it is preferable that the light source is a combination of monochromatic LEDs which can obtain a specific wavelength range with strong emission strength.

Moreover, the apparatus for facial treatment and hair restoration of a first aspect of the present invention include the light source means includes a plurality of monochromatic laser diode light sources having different peak wavelengths and a bandpass filter for focusing wavelength bands of each light emitted from the monochromatic laser diode light sources so that the light has a half bandwidth of 10 nm or less.

Moreover, the apparatus for facial treatment and hair restoration of a first aspect of the present invention include the light source means includes the light source means is a monochromatic laser diode light source or a laser light source which can irradiate a monochromatic light of an ultra narrow bandwidth having a half bandwidth of 10 nm or less.

Here, specifically, the diffusion means of the apparatus for facial treatment and hair restoration of the first aspect uses one or two or more from among a cylindrical lens, a diffuser panel, and a diffusion lens. In a case where the monochromatic LED light source is used, the diffusion lens is preferable from the viewpoint of suppressing a decrease in light volume to the minimum extent possible. Further, in a case where a single laser diode light source is used, from a viewpoint of diffusing a light having strong directional characteristics to a large area, it is preferable that the diffusion means uses one or two or more lenses from among a cylindrical lens, a diffuser panel, and a diffusion lens.

Further, in a case where a laser light source or a laser diode light source is used, the light emitted from the light source is a light having an ultra narrow bandwidth. However, the light has strong directional characteristics by itself and cannot be irradiated on a wide area such as the scalp or face. Therefore, the light emitted from the light source is diffused by the diffusion means to be irradiated onto a wide area such as the scalp or face. Especially, in a case where the laser light source is used, the light has a strong energy and there may be a case where the skin is damaged due to the heat of the energy if the light is directly irradiated onto the skin. Therefore, the energy is sufficiently weakened by diffusion. However, in a case where the light source is a laser diode and the bandwidth of the light is not the ultra narrow bandwidth, the bandwidth is changed by use of a bandpass filter so that the bandwidth becomes the ultra narrow bandwidth having a half bandwidth of 10 nm or less.

Here, in a case where a cylindrical lens is used as the diffusion means, two cylindrical lenses are provided in positions orthogonal to each other. Generally, the cylindrical lens is used when adjustment of magnification in only one direction is required and the lens forms an image in one direction. However, because the same image forming formula as that of a spherical lens is established, a beam is collimated in one direction by a first cylindrical lens and then is collimated by a second cylindrical lens in a direction that is orthogonal to the first one. Here, a diffusion lens, a diffuser panel or the like may be further used with the cylindrical lens.

Next, the system for facial treatment and hair restoration of a first aspect of the present invention include the above-mentioned apparatus for facial treatment and hair restoration; a colorless transparent silicon thin film for covering skin of a thin hair cephalic part or a part or skin of the face; and an iontophoresis means for carrying out iontophoresis by applying gel having effective ingredients between the silicon thin film and surface of the skin; wherein the system can activate cells of skin and under the skin by irradiating a monochromatic light of an ultra narrow bandwidth having a half bandwidth of 10 nm or less by use of the apparatus for facial treatment and hair restoration from above the silicon thin film in a condition where effective ingredients are caused to permeate under the skin by use of the iontophoresis means.

According to the above-mentioned configuration, in addition to iontophoresis generally carried out in the esthetic industry, activation of cells of the cephalic part and other skin can be carried out by covering a target part with a colorless transparent silicon thin film and irradiating an ultra narrow band monochromatic visible light having a half bandwidth of 10 nm or less so that hair restoration or hair growth is enhanced.

Further, the colorless transparent silicon thin film is used to enable irradiation of the visible light from above the thin film by the apparatus for facial treatment and hair restoration of the present invention.

Next, the system for facial treatment and hair restoration of a second aspect of the present invention include the above-mentioned apparatus for facial treatment and hair restoration; a colorless transparent silicon thin film for covering skin of a thin hair cephalic part or a part or skin of face; an iontophoresis means for carrying out iontophoresis by applying gel having effective ingredients between the silicon thin film and surface of skin; and a sonophoresis means for causing ultrasonic vibrations to the silicon thin film; wherein the system can activate cells of skin and under the skin by irradiating a monochromatic light of an ultra narrow bandwidth having a half bandwidth of 10 nm or less by use of the apparatus for facial treatment and hair restoration from above the silicon thin film in a condition where effective ingredients are caused to permeate under the skin by use of the iontophoresis means and the sonophoresis means.

According to the above-mentioned configuration, iontophoresis and sonophoresis can be simultaneously carried out. Therefore, it becomes possible to cause effective properties to be absorbed into the skin efficient and to shorten treatment time. Here, the ultrasonic vibration may be carried out before or after the iontophoresis. In this case, although treatment time is not shortened, it becomes possible to efficiently cause the effective ingredients to be similarly absorbed into the skin.

Here, it is preferable that microparticles of at least one type selected from gold, silver, platinum, and palladium is mixed in the silicon thin film when the thin film is formed.

Moreover, it is preferable that the silicon thin film has the shape of a human face and includes a part which is formed to have openings at portions corresponding to eyes, nose, and mouth and a part which is approximately trapezoidal and for covering the opening portion corresponding to the nose.

### Effects of the Invention

According to the apparatus for facial treatment and hair restoration of the present invention, such an effect is brought about that interference by the light of the light source is significantly reduced and only light in a narrow bandwidth, which is the bandwidth region of the monochromatic LED light source and is in the vicinity of the peak bandwidth, is extracted and used, thereby a stronger effect can be obtained compared to a case where the same amount of light having a similar bandwidth region is used. That is, original characteristics of the selected light of the narrow bandwidth can be utilized for beauty.

Moreover, according to the apparatus for facial treatment and hair restoration of the present invention, adjustment of a monochromatic light in a narrow bandwidth having a half bandwidth of 10 nm or less (visible light range) ultimately close to nonstep adjustment can be carried out. Including monochromatic LED light sources having a slightly different peak wavelength increases steps for switching and when the peak wavelength exists in the visible light range, strength of the light can be sufficiently ensured, types of selectable narrow bandwidths are increased, and characteristics of the visible light in each of the ultra narrow bandwidths can be used to the maximum extent possible.

According to the system for facial treatment and hair restoration of the present invention, in addition to the iontophoresis which is generally carried out, irradiation of visible light of the ultra narrow bandwidth having a half bandwidth of 10 nm or less the waveband of which is narrowed down is carried out to activate cells in and under the skin of scalp and face so that hair restoration or hair growth is enhanced, skin is beautified, or facial treatment is performed.

### BRIEF DESCRIPTION OF THE DRAWINGS

FIG. 1 Illustrates the implementation of example 1 of a diagram of a facial hair-growth device (a) is aside sectional view, (b) is the front elevation

FIG. 2 Illustrates the implementation of example 1 of an irradiation unit of the apparatus of facial hair growth device in LED light source configuration diagram

FIG. 3-1 Illustrates the full-face photo taken before 1st irradiation by LED

FIG. 3-2 Illustrates the full-face photo taken before 1st irradiation by LED (Depicts the Line)

FIG. 3-3 Illustrates the full-face photo taken after 1st irradiation by LED

FIG. 3-4 Illustrates the full-face photo taken after 1st irradiation by LED (Depicts the Line)

FIG. 3-5 Illustrates the full-face photo taken before second irradiation by LED

FIG. 3-6 Illustrates the full-face photo taken before second irradiation by LED (Depicts the Line)

FIG. 3-7 Illustrates the full-face photo taken after second irradiation by LED

FIG. 3-8 Illustrates the full-face photo taken after second irradiation by LED (depicts the line)

FIG. 4 Illustrates the implementation of example 1 of facial hair-growth device when the use of photos

FIG. 5-1 Illustrate a change illustration of male monitor 1 in hair growth

FIG. 5-2 Illustrate a change illustration of male monitor 2 in hair growth

FIG. 5-3 Illustrate a change illustration of male monitor 3 in hair growth

FIG. 5-4 Illustrate a change illustration of male monitor 4 in hair growth

FIG. 5-5 Illustrate a change illustration of male monitor 5 in hair growth

FIG. 5-6 Illustrate a change illustration of male monitor 6 in hair growth

FIG. 5-7 Illustrate a change illustration of male monitor 7 in hair growth

FIG. 5-8 Illustrate a change illustration of male monitor 8 in hair growth

FIG. 5-9 Illustrate a change illustration of male monitor 9 in hair growth

FIG. 5-10 Illustrate a change illustration of male monitor 10 in hair growth

FIG. 5-11 Illustrate a change illustration of male monitor 11 in hair growth

FIG. 6 Illustrate a changer illustration of male monitor 4 that used the conventional equipment.

FIG. 7 Illustrate a image use of the facial hair growth device

FIG. 8 Illustrate the illustration about implementation of example 2 of the facial hair growth device.

FIG. 9 Illustrate the illustration about implementation of example 3 of the facial hair growth device.

FIG. 10 Illustrate the schematic diagram about implementation of Example 4 of the facial hair growths System.

FIG. 11 Illustrate Mask that was molded in silicon laced with gold particles

### DESCRIPTION OF SYMBOLS

1 facial hair growth
2 Monochromatic LED light source unit
3 Guiding light path
4 Spectrometer
5 Lens
6 Grating
7 LED light source
10 Focusing lens
11 Slit
12 Diffusion Lens
13 Band-pass filter
14 Power cable/signals
15 Chassis frame
16 Red LED light source
17 Blue LED light source
18 Green LED light source
20 The Specific visible spectrum,
21 Human face
51 Iontophoretic device
52 Silicon thin film
53 Visible light
54 The head
60 Mask
61 Silicon thin film covering the area of the nose
62 Gold particles

### BEST MODE FOR CARRYING OUT THE INVENTION

Embodiments of the present invention will be described in detail below with reference to the drawings.

### EXAMPLE 1

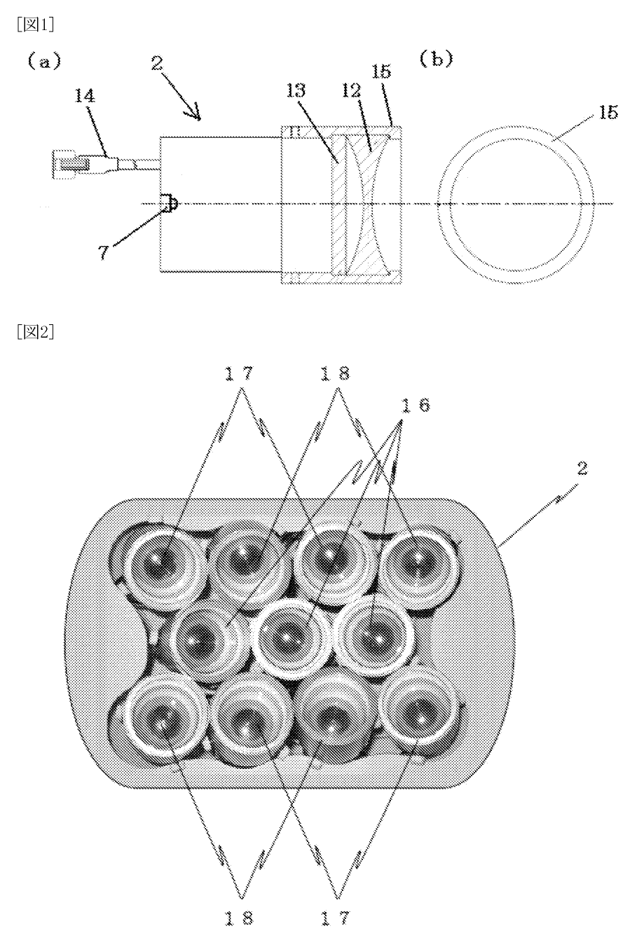

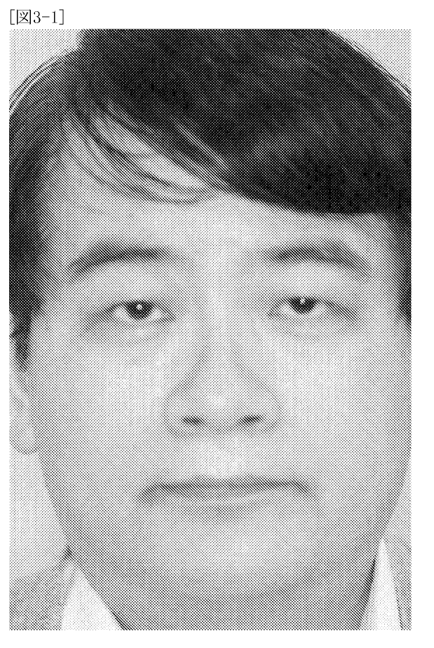

FIG. 1 shows a configuration diagram of an apparatus for facial treatment and hair restoration according to a first embodiment. The apparatus for facial treatment and hair restoration of the first embodiment includes three monochromatic LED light sources 7 (red color LED light source, blue color LED light source, and green color LED light source) which have a peak wavelength and half bandwidth characteristics shown in the following Table 1, a bandpass filter 13 for narrowing wavelength bands of each light emitted from the monochromatic light sources 7 down, and a diffusion lens 12 for diffusing the light of an ultra narrow bandwidth having a half bandwidth of 10 nm which transmitted the bandpass filter 13.

Here, the monochromatic LED light source 7 may include a parallel light LED light source IBF-LS of IMAC CO., Ltd. The light irradiated from the monochromatic light source 7 is an ultra-long cast light with directionality. Diameter of the light is approximately 5 cm and this light is diffused via the diffusion lens 12.

TABLE 1

| | peak | halfwidth | The use of |
|---|---|---|---|
| light source | wavelength(nm) | (nm) | the number |
| red LED light source | 627 | 20 | 3 |
| blue LED light source | 470 | 25 | 4 |
| green LED light source | 530 | 35 | 4 |

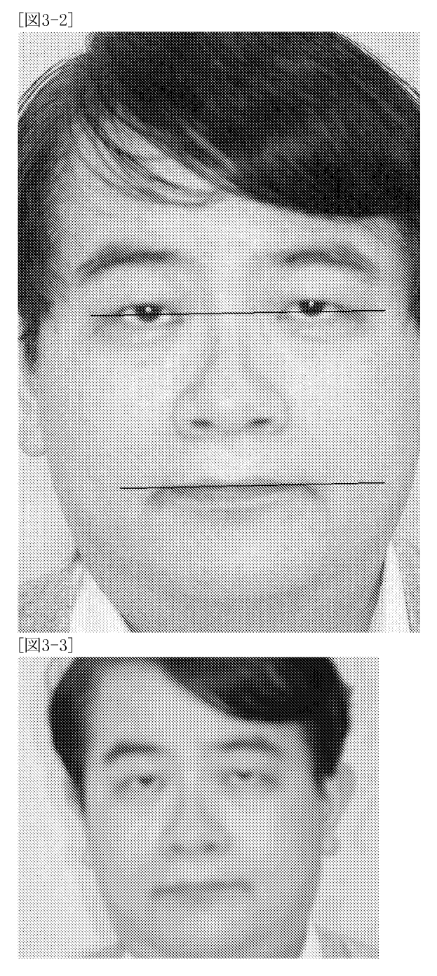

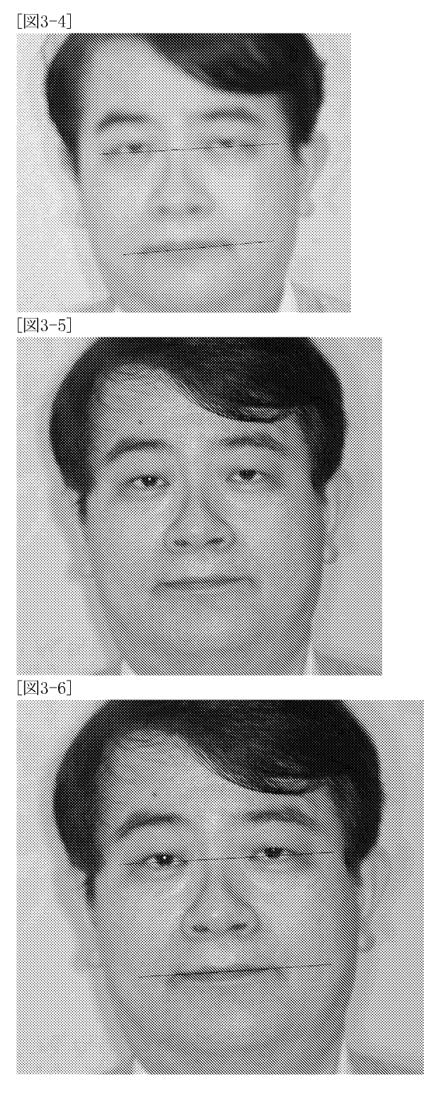

These light sources are combined for the number used in Table 1 to configure an emission unit of the apparatus for facial treatment and hair restoration of Embodiment 1. FIG. 2 shows a block diagram showing a configuration of the LED light sources of the emission unit of the apparatus for facial treatment and hair restoration of Embodiment 1. As shown in FIG. 2, light sources of red color LEDs, blue color LEDs, and green color LEDS are disposed. Three red color LEDs are continuously disposed at the center in a row and four blue color LEDs and four green color LEDs surround the periphery thereof. Specifically, two blue color LEDs and two green color LEDs are provided on the left and right sides of the red color LEDs to sandwich the three red color LEDs at the center. The blue color LEDs and the green color LEDs are disposed so as to face each other and alternately. Moreover, light axis of the blue color LEDs and the green color LEDs are respectively inclined toward the center so that all the LEDs irradiate light to a certain region.

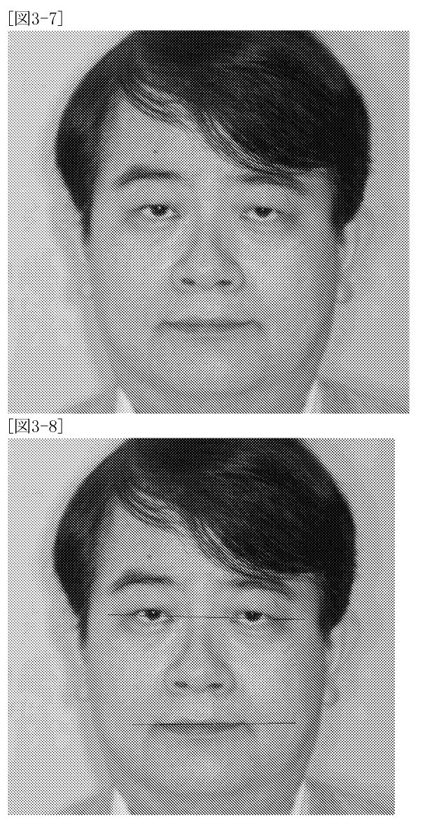

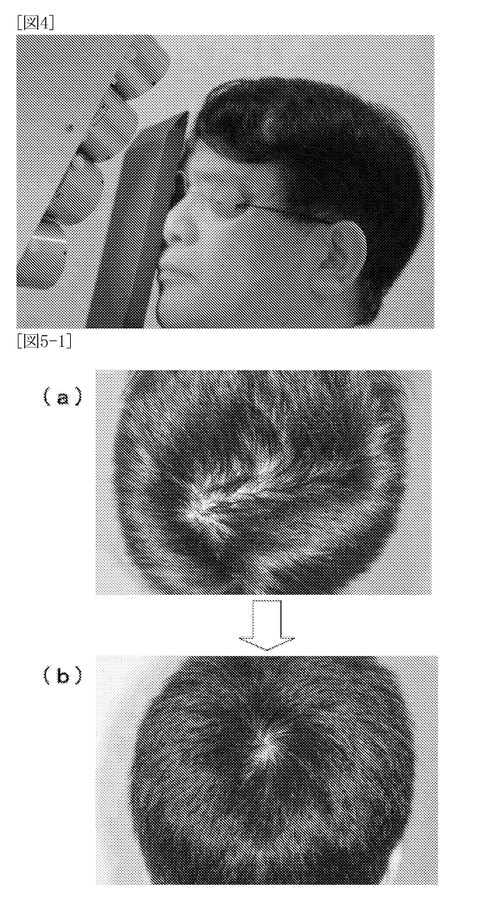

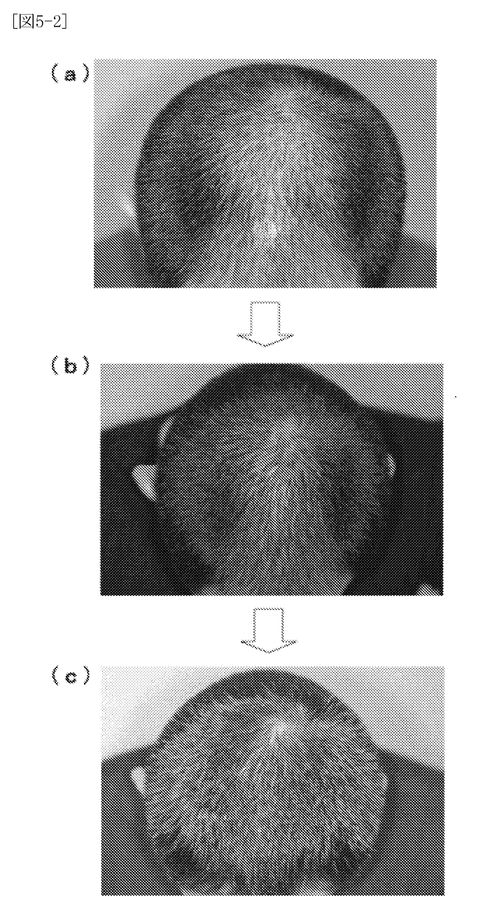

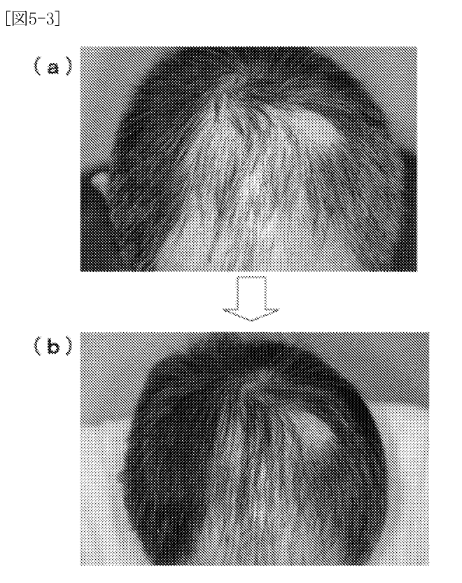

Next, the facial treatment effect in a case where a light of an ultra narrow bandwidth having a half bandwidth of 10 nm is diffused and irradiated by the apparatus for facial treatment and hair restoration of the first embodiment onto the scalp is explained with reference to photos and other data. An experiment was conducted on a 51-year-old man. The apparatus for facial treatment and hair restoration of Embodiment 1 was used on the left side of the man's face. Photos of the man's face before and after use are shown in FIGS. 3-1 to 3-8. Here, in FIG. 3-2, FIG. 3-4, FIG. 3-6, and FIG. 3-8, a line connecting the outer creases of both eyes and a line connecting the creases of the lips are drawn to show the effect of the apparatus for facial treatment and hair restoration. Moreover, FIG. 4 shows a case where the apparatus for facial treatment and hair restoration of Embodiment 1 is being used on the left side of the man's face.

Procedures of the experiment and photos showing the process of the experiment are as follows:

### (1) First Day (Jan. 6, 2007)

FIG. 3-1 and FIG. 3-2 show the face of the man taken before first irradiation of the LED. When observed carefully, it is understood that the line connecting the outer creases of both eyes and a line connecting the creases of the lips are parallel and therefore, there is no difference between the left side and right side of the man's face.

The apparatus for facial treatment and hair restoration of Embodiment 1 was used according to the following procedures on the first day.

Only the left side of the face was irradiated by the red color LED light source for 10 minutes.

Only the left side of the face was irradiated by the green color LED light source for 10 minutes.

FIG. 3-3 and FIG. 3-4 show photos of the man's face taken after the first LED irradiation. Unfortunately, the photos are slightly out of focus, but the photos in FIGS. 3-3 and 3-4 after the first irradiation show that the outer crease of man's left eye droops downward slightly while the left side of his lips are elevated slightly when compared to the photos before the first LED irradiation. This can be understood when FIG. 3-4 is observed. In the photo, a line connecting the outer creases of both eyes is a downward sloping line while a line connecting the creases of the lips is diagonally right up. Therefore, it is understood that tightening effects appeared only on the left side of the man's face by the above-mentioned irradiation.

### (2) Second Day (Jan. 7, 2007)

Photos taken before the second LED irradiation which was conducted 24 hours after irradiation on the first day are shown in FIG. 3-5 and FIG. 3-6. It is understood from FIG. 3-5 and FIG. 3-6 that tightening effects by the irradiation on the first day still remain on the man's face.

The apparatus for facial treatment and hair restoration of a third embodiment was used according to the following procedures on the second day.

Only the left side of the face was irradiated by the red color LED light source for 10 minutes.

Only the left side of the face was irradiated by the green color LED light source for 10 minutes.

FIG. 3-7 and FIG. 3-8 show photos of the man's face taken after the second LED irradiation. It is understood that the outer crease of the man's left eye droops downward further while the left side of his lips are elevated after the second LED irradiation from FIG. 3-7 and FIG. 3-8. This can be understood when FIG. 3-8 is observed. In the photo, the line connecting the outer creases of both eyes is a downward sloping line while the line connecting the creases of the lips is diagonally right up. Therefore, it is understood that tightening effects strongly appeared only on the left side of the man's face by the above-mentioned irradiation.

Next, effects of hair growth in a case where a light of an ultra narrow bandwidth having a half bandwidth of 10 nm is diffused and irradiated by the apparatus for facial treatment and hair restoration of Embodiment 1 onto the scalp is explained with reference to photos and other data.

Here, for the purpose of confirming that irradiation of a light having a narrow bandwidth spectrum strongly effects activation of cells of a living body, hair growth effects of red, green, and blue narrow bandwidth lights are confirmed. As subjects, 11 male monitors aged between 29 and 59 (average age 48.1 years old) with confirmed hair-thinning on their scalps and were subjected to irradiation once a week on average for three months or more were prepared.

As an experiment method, a device was developed that makes high-intensity LEDs of three colors, red, blue, and green, emit parallel light and can irradiate a light having a narrow bandwidth spectrum of a half bandwidth of 10 nm by use of a bandpass filter and a diffusion lens. In terms of the amount of light, three red color light source units and respective four blue color and green color light source units respectively for green color and blue color were prepared. A total of 11 light source units were arranged on a plane surface, setting light axis of the unit slightly leaning to the center. Then, the light was irradiated to the scalp of the monitors from about 10 cm away by the order of red, blue, and green for 7 minutes for each color and a total of 21 minutes. Irradiation was carried out once to three times a week for each monitor (once a week or more on average) and the experiment period was between three and eight months. The monitors did not take any special treatment for their scalps such as use of a hair growing agent and unclogging of pores and carried out their normal daily routines. To determine the effects, the M-shaped thin hair part was excluded. Determination was conducted according to the criteria 1 to 3 in the following Table 2.

TABLE 2

| | Determination | Expression | Condition |
|---|---|---|---|
| 1 | Effective | Ia | 25% or more of region where skin could be seen due to |
| | | | thinning hair was reduced |
| | | Ib | Hair was visibly strong or hair was visibly straightened |
| 2 | Very effective | IIa | 50% or more of region where skin could be seen due to |
| | | | thinning hair was reduced |
| | | IIb | Hair was visibly, significantly stronger or hair was |
| | | | significantly straightened |
| 3 | Healed | IIIa | Condition of scalp returning approximately to a condition |
| | | | which is before hair started thinning |

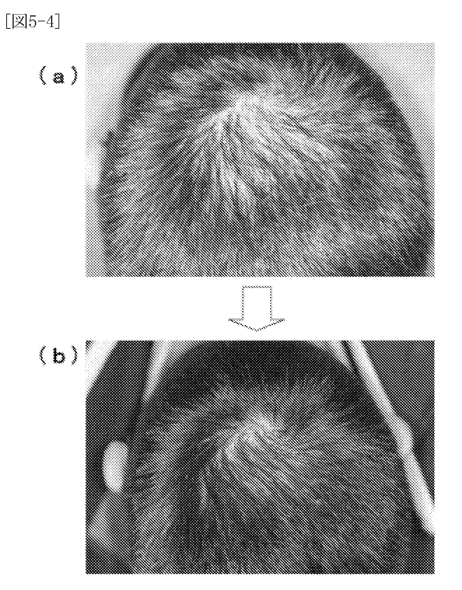

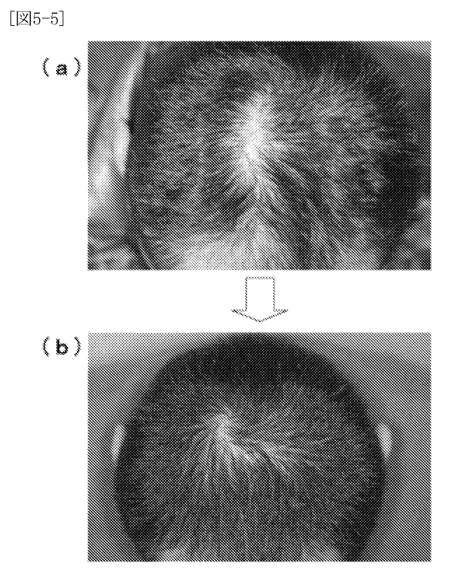

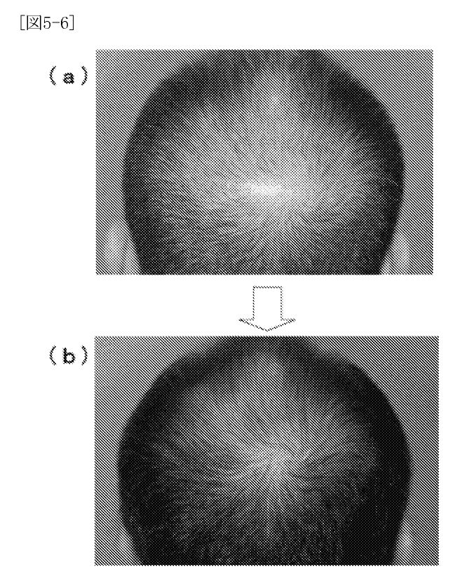

TABLE 3

| | Clinical | | Hamilton | | | |
|---|---|---|---|---|---|---|
| No. | trials | Age | classification | Hair treatment | Photographs of the treatment process | |
| A | monitor 1 | 38 | IIIvertex Type | Recovery (IIIa) | FIG. 5-1 | (a) Pre-treatment |
| | | | | | | (b) After 21 weeks (29 times of Irradiation) |
| B | monitor 2 | 29 | Va Type | Recovery (IIIa) | FIG. 5-2 | (a) After 8 weeks (15 times of Irradiation) |
| | | | | | | (b) After 18 weeks (33 times of Irradiation) |
| | | | | | | (c) After 26 weeks (50 times of Irradiation) |
| C | monitor 3 | 55 | VI Type | Markedly (IIa) | FIG. 5-3 | (a) Pre-treatment |
| | | | | | | (b) After 17 weeks (32 times of Irradiation) |
| D | monitor 4 | 59 | IIIvertex Type | Markedly (IIa) | FIG. 5-4 | (a) Pre-treatment |
| | | | | | | (b) After 18 weeks (37 times of Irradiation) |
| E | monitor 5 | 38 | IIIvertexn Type | Markedly (IIa) | FIG. 5-5 | (a) Pre-treatment |
| | | | | | | (b) After 9 weeks (20 times of Irradiation) |
| F | monitor 6 | 50 | VI Type | Markedly (IIa) | FIG. 5-6 | (a) Pre-treatment |
| | | | | | | (b) After 27 weeks (41 times of Irradiation) |
| G | monitor 7 | 58 | V Type | Markedly (IIa) | FIG. 5-7 | (a) Pre-treatment |
| | | | | | | (b) After 11 weeks (14 times of Irradiation) |
| H | monitor 8 | 45 | VI Type | Markedly (IIb) | FIG. 5-8 | (a) Pre-treatment |
| | | | | | | (b) After 19 weeks (19 times of Irradiation) |
| I | monitor 9 | 54 | VI Type | Effective (Ib) | FIG. 5-9 | (a) Pre-treatment |
| | | | | | | (b) After 33 weeks (80 times of Irradiation) |
| J | monitor 10 | 55 | V Type | Markedly (IIa) | FIG. 5-10 | (a) Pre-treatment |
| | | | | | | (b) After 54 weeks (128 times of Irradiation) |
| K | monitor 11 | 47 | IV Type | Markedly (IIa) | FIG. 5-11 | (a) Pre-treatment |
| | | | | | | (b) After 31 weeks (41 times of Irradiation) |

The transition result of hair restoration in man 1-11 of the monitor is as shown in table 3 listed below.

Moreover, a result of a case where hair growth effects were researched using a conventional apparatus, which is a combination of monochromatic LEDs but does not emit a narrow bandwidth light, with respect to the above-mentioned monitor 4 is shown in FIG. 6. However, this interactive effect was conducted when monitor 4 was in a thinning hair condition about one year before the experiment of the above-mentioned Table 2 (about one year before the photo shown in FIG. 5-4 (a)). FIG. 6 (a) is a condition before using the conventional apparatus and FIG. 6 (b) is a condition after irradiation was carried out 31 times in 12 weeks. It is confirmed from FIGS. 6 (a) and (b) that there is no visible change when the conventional apparatus in which monochromatic LEDS are combined was used. This implies a strong effect of the narrow bandwidth light.

In a case where irradiation was carried out once a week or more, the efficacy rate was 100%. Among them, thinning hair of two (monitors 1 and 2) healed. Eight of the other monitors showed very effective results and one (monitor 9) showed an effective result.

As a result of the above, all of the 11 monitors confirmed hair growth and the efficacy rate was 100%, while nine out of the 11 confirmed very effective results or more and two out of 11 confirmed restoration. In many cases, the effects appeared by the order of reduced hair loss, strength in hair and appearance of straightened hair, regeneration of hair whorl and visible hair growth (visible change from downy hair or vellus hair to strong hair). Moreover, little effect could be seen for M-shaped thin hair. In all of the above-mentioned 11 examples, effects could be confirmed visually within a few months.

From the above results, it was confirmed that irradiation of narrow bandwidth light in a visible light region by use of the apparatus for facial treatment and hair restoration of Embodiment 1 has a strong hair growth effect and it was suggested that the narrow bandwidth light strongly activates living cells. Moreover, strong pressure effect, reduction of wrinkles and shadows, significant improvement in makeup, and the like by the irradiation of the narrow bandwidth light by the apparatus for facial treatment and hair restoration of Embodiment 1 were also confirmed. Thus, it is understood that irradiation of the narrow bandwidth light will play a great role in hair growth treatment, facial treatment, and the like. Moreover, effects such as an increase in fibroblasts by red color light or sterilization of acne by blue color light have been discussed and determining the specific effects on living cells or the like by each wavelength is anticipated. If effects of each wavelength will be made clear, superior combination treatment by irradiating lights of more effective wavelengths or a plurality of wavelengths for hair growth, facial treatment, or other dermatological diseases can be made clear.

To summarize the above, first of all, every wavelength of a visible light has a unique effect on a living cell or the like and the effect gradually changes as the wavelength varies. Moreover, it can be said that there is a possibility that the more wavelengths vary, the more wavelengths negate the effect that each of the wavelengths brings to a living cell or the like. Further, it can be said that an effect unique to the central wavelength of a narrow bandwidth light having a smaller half bandwidth (having narrower width of wavelength range) becomes stronger if the volume of the light is constant.

### EXAMPLE 2

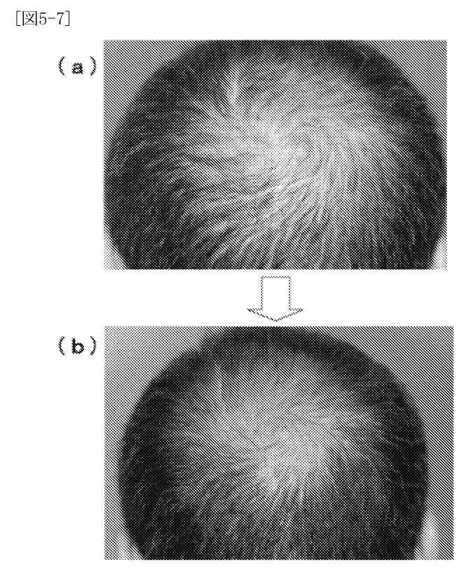

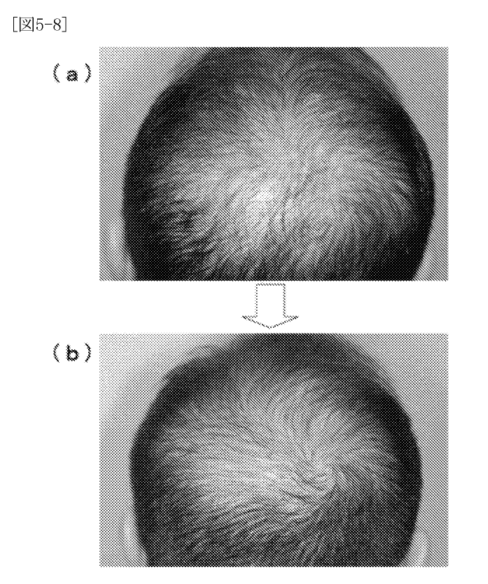

FIG. 7 shows a usage image of the apparatus for facial treatment and hair restoration. FIG. 7 shows a case where the apparatus for facial treatment and hair restoration 1 is used for a face. In the case that the apparatus is used for a scalp, a visible light 20 having a specific waveband is irradiated onto the scalp.

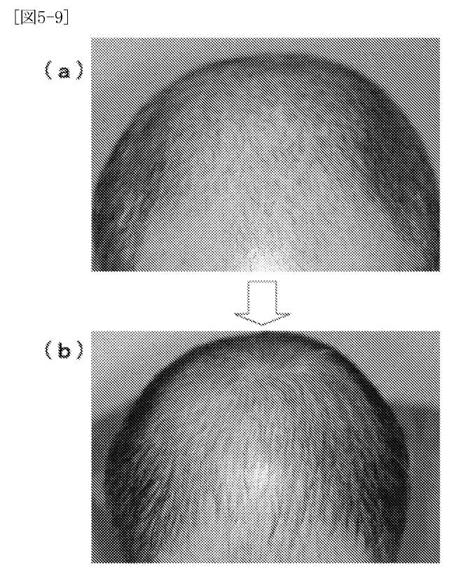

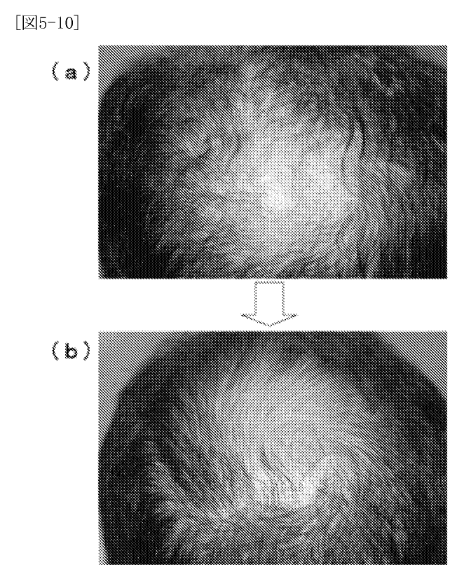

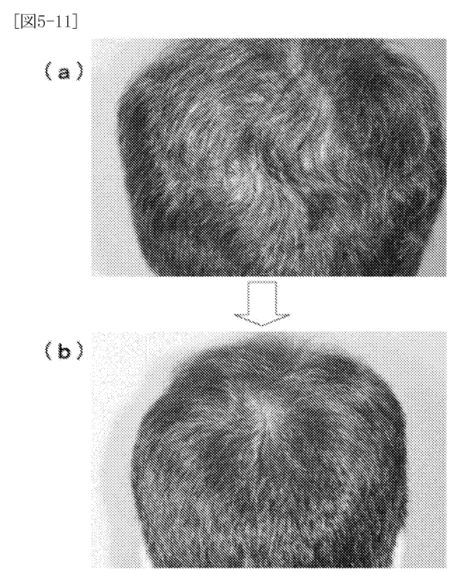

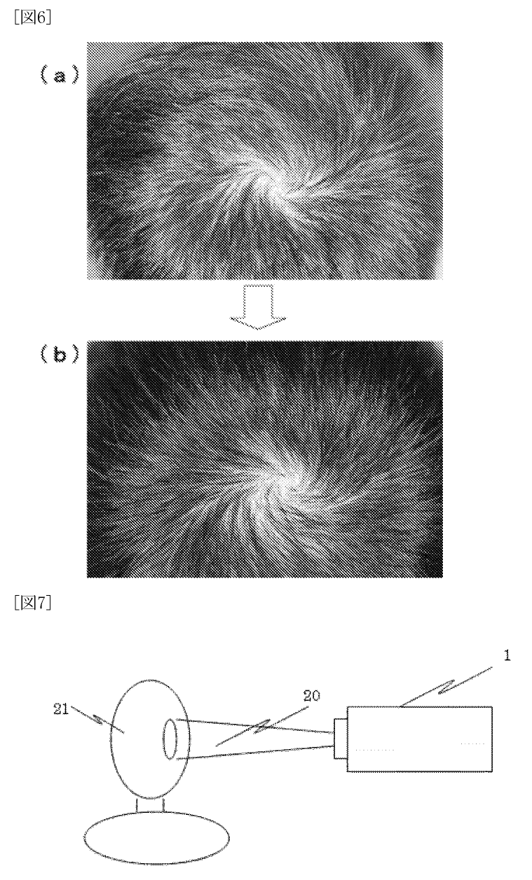

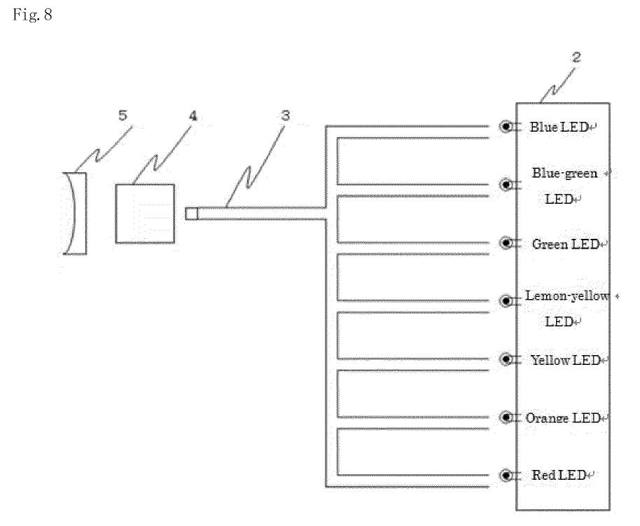

FIG. 8 shows a configuration diagram of an apparatus for facial treatment and hair restoration according to an embodiment 2. The apparatus for facial treatment and hair restoration of Embodiment 2 includes a monochromatic LED light source unit 2 having a plurality of monochromatic LED light sources which are arranged on a substrate, a light guiding path 3 for guiding monochromatic lights emitted from each of the monochromatic LED light sources in a manner that the lights are bundled (specifically, a bundle of optical fibers is used), a spectroscope 4 for dispersing the light emitted from the light guiding path 3, and a diffusion lens 5 provided in front of the spectroscope 4. The diffusion lens 5 includes a concave lens or the like and is for diffusing a light so that the light can be irradiated onto an irradiation target such as a face in an appropriate size.

### EXAMPLE 3

FIG. 9 shows a configuration diagram of an apparatus for facial treatment and hair restoration according to an embodiment 3. The apparatus for facial treatment and hair restoration of Embodiment 3 includes a monochromatic LED light source unit 2 having a plurality of monochromatic LED light sources which are arranged on a substrate, a focusing lens 10 provided in front of each of the monochromatic LEDs 2 (in light irradiation direction), slits 11 provided in front of the focusing lens 10, and a diffusion lens 12 provided in front of the slits 11.

Here, the focusing lens 10 includes a combination of convex lenses and is for focusing a light made incident into the slits 11 provided in front of the lens. Moreover, the diffusion lens 12 includes a concave lens or the like and is for diffusing a light so that the light can be irradiated onto an irradiation target such as a face in an appropriate size. Further, in FIG. 9, a plurality of the monochromatic LEDs 2 are provided two-dimensionally in a matrix state in the horizontal direction and in a direction perpendicular to the image plane of the figure itself with a certain pitch. At the same time, the LEDs 2 are provided parallel to each other so that the LEDs 2 can emit lights to the left side in the figure.

The slits 11 are formed with the same pitch as the monochromatic LED light source and each of the slits 11 has the same width (width in the diffusion direction). As a result thereof, lights having the same wavelength band pass through all the slits 11. The slits 11 are enabled to move in an irradiation direction and a direction perpendicular to the irradiation direction by a drive means (not shown). Then, if the slits 11 are moved in the irradiation direction, spectrum position corresponding to the slits 11 changes along with the move and therefore diffused lights in a wavelength band having different central wavelengths pass through the slits 11. Thus, it becomes possible to more appropriately select a wavelength band (central wavelength) of a light to be irradiated.

### EXAMPLE 4

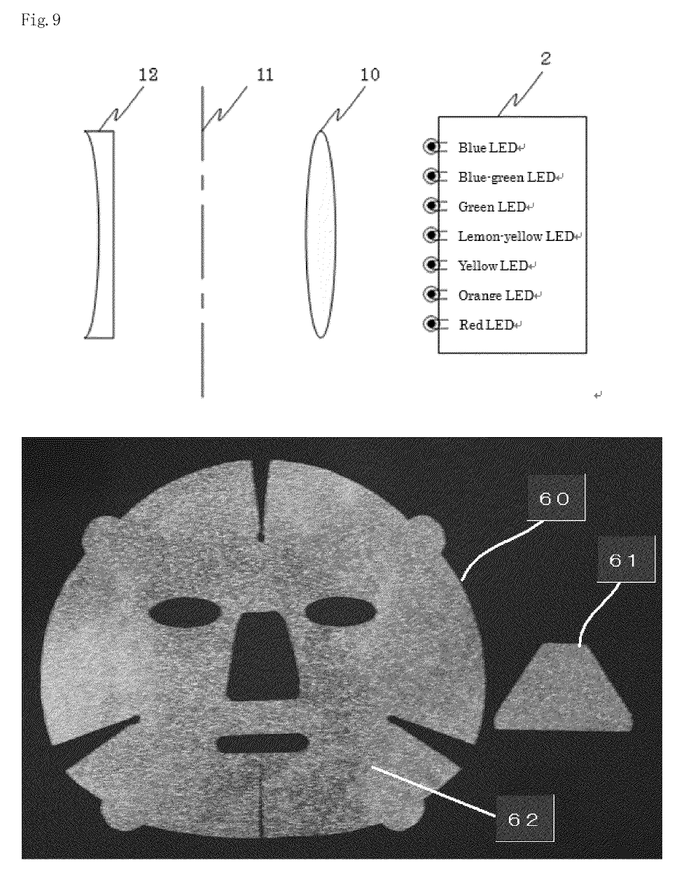

FIG. 10 shows a system configuration of a system for facial treatment and hair restoration of an embodiment 4. A commercially available iontophoresis device 1 is used as an iontophoresis means. For a colorless transparent silicon thin film 2, an industrial silicon having quite high transparency is used and when the silicon thin film is formed, microparticles of gold are mixed in. The colorless transparent silicon thin film 2 has a superior flexibility and matches the shape of a head if the silicon thin film is caused to cover a cephalic part 4 of a human and fits the cephalic part 4 so as to suppress the cephalic part.

The colorless transparent silicon thin film 2 may cover all of the cephalic part or may cover part of the cephalic part. It is possible to adjust a part to be covered depending on the symptoms.

Procedures are as follows: Liquid or gel including ingredients effective for hair growth is applied to a portion where restoration of hair is requested and the portion is covered with the silicon thin film 2. Then, by use of clips attached to a polarization code of the iontophoresis device 1, four corners of the colorless transparent silicon thin film 2 are tucked in while another pole is brought into contact with an arm or the like. Gel or the like under the colorless transparent silicon thin film 2 functions as one pole of iontophoresis electrodes of the iontophoresis device 1.

Moreover, in the above-mentioned condition, a visible light 53 is irradiated from above the cephalic part by use of the apparatus for facial treatment and hair restoration 1 of Embodiment 1. The apparatus for facial treatment and hair restoration 1 emits a monochromatic light in an ultra narrow bandwidth having a half bandwidth of 10 nm or less and diffuses the light to an irradiation area of the cephalic part by a diffusion lens.

### EXAMPLE 5

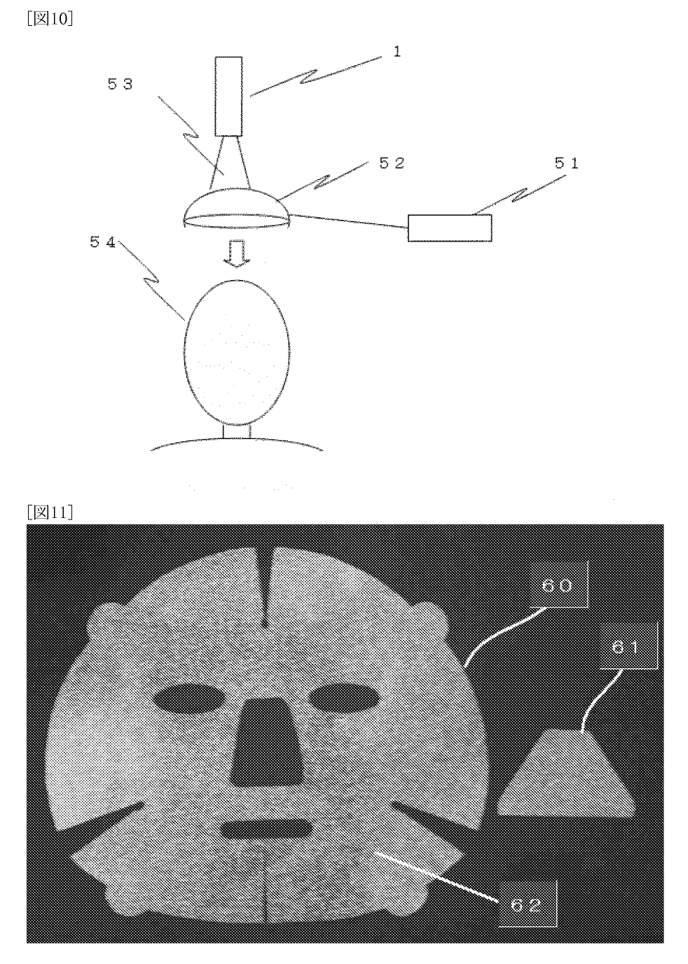

FIG. 11 shows a mask 60 and a trapezoidal silicon thin film for covering a nose part formed with the silicon thin film in which gold microparticles 62 are mixed. An industrial silicon having quite high transparency is used for the silicon thin film. Moreover, gold powder is used as the gold microparticle 62 mixed into the silicon thin film at the time of forming. As shown in FIG. 11, holes are created in the mask 60 at the parts that correspond to the nose or mouth of a human face to avoid trouble in breathing by a subject.

### INDUSTRIAL APPLICABILITY

Other than combinations with various types of face lotions or beauty lotions or combinations with iontophoresis or sonophoresis, the apparatus for facial treatment and hair restoration according to the present invention may be combined with irradiation of far infrared rays or irradiation of negative ions or combined with pulse static electromagnetic field to be used in medical treatment or aesthetic industry such as enhancing hair restoration in the cephalic part, beautification of skin, or facial treatment. Moreover, effects of the apparatus for facial treatment and hair restoration according to the present invention may be used for enhancement of growth of agricultural products such as plants or for growth of livestock.

The system for facial treatment and hair restoration according to the present invention can enhance hair growth or hair restoration by efficiently activating cells of skin and cells under the skin of cephalic part or the like for a patient who has less hair in the cephalic part due to aging, disease, or the like. Therefore, the present invention is useful for medical treatment or the aesthetic industry such as treatment for restoration or growth of hair, or for the wig industry.

1. An apparatus for facial treatment and hair restoration comprising:
a light source means which can emit a monochromatic light of an ultra narrow bandwidth having a half bandwidth of 10 nm or less; and
a diffusion means which can diffuse the monochromatic light of an ultra narrow bandwidth emitted from the light source means so that an area for irradiation can be increased.

2. The apparatus for facial treatment and hair restoration according to claim 1 further comprising a peak wavelength variation means for enabling to vary a peak wavelength of the monochromatic light.

3. The apparatus for facial treatment and hair restoration according to claim 2, wherein
the peak wavelength variation means can vary the peak wavelength of the monochromatic light in a red or orange wavelength range.

4. The apparatus for facial treatment and hair restoration according to claim 2, wherein
the peak wavelength variation means can vary the peak wavelength of the monochromatic light in a green wavelength range.

5. The apparatus for facial treatment and hair restoration according to claim 2, wherein
the peak wavelength variation means can vary the peak wavelength of the monochromatic light in a blue wavelength range.

6. The apparatus for facial treatment and hair restoration according to claim 1, wherein
the light source means includes a plurality of monochromatic light emitting diode light sources having different peak wavelengths and a bandpass filter for focusing wavelength bands of each light emitted from the monochromatic light emitting diode light sources so that the light has a half bandwidth of 10 nm or less.

7. The apparatus for facial treatment and hair restoration according to claim 1, wherein
the light source means includes a plurality of monochromatic laser diode light sources having different peak wavelengths and a bandpass filter for focusing wavelength bands of each light emitted from the monochromatic laser diode light sources so that the light has a half bandwidth of 10 nm or less.

8. The apparatus for facial treatment and hair restoration according to claim 1, wherein
the light source means is a monochromatic laser diode light source or a laser light source which can irradiate a monochromatic light of an ultra narrow bandwidth having a half bandwidth of 10 nm or less.

9. The apparatus for facial treatment and hair restoration according to claim 1, wherein
the diffusion means is any one or a combination of two or more of a cylindrical lens, a diffuser panel, and a diffusion lens.

10. A system for facial treatment and hair restoration comprising:
the apparatus for facial treatment and hair restoration according to claim 1;
a colorless transparent silicon thin film for covering skin of a thin hair cephalic part or a part or skin of the face; and
an iontophoresis means for carrying out iontophoresis by applying gel having effective ingredients between the silicon thin film and surface of the skin; wherein
the system can activate cells of skin and under the skin by irradiating a monochromatic light of an ultra narrow bandwidth having a half bandwidth of 10 nm or less by use of the apparatus for facial treatment and hair restoration from above the silicon thin film in a condition where effective ingredients are caused to permeate under the skin by use of the iontophoresis means.

11. A system for facial treatment and hair restoration comprising:
the apparatus for facial treatment and hair restoration according to claim 1;
a colorless transparent silicon thin film for covering skin of a thin hair cephalic part or a part or skin of face;
an iontophoresis means for carrying out iontophoresis by applying gel having effective ingredients between the silicon thin film and surface of skin; and
a sonophoresis means for causing ultrasonic vibrations to the silicon thin film; wherein
the system can activate cells of skin and under the skin by irradiating a monochromatic light of an ultra narrow bandwidth having a half bandwidth of 10 nm or less by use of the apparatus for facial treatment and hair restoration from above the silicon thin film in a condition where effective ingredients are caused to permeate under the skin by use of the iontophoresis means and the sonophoresis means.

12. The system for facial treatment and hair restoration according to claim 10, wherein
microparticles of at least one type selected from gold, silver, platinum, and palladium is mixed in the silicon thin film when the thin film is formed.

13. The system for facial treatment and hair restoration according to claim 10, wherein the silicon thin film has the shape of a human face and includes a part which is formed to have openings at portions corresponding to eyes, nose, and mouth and a part which is approximately trapezoidal and for covering the opening portion corresponding to the nose.
